(19) United States
(12) Patent Application Publication
(10) Pub. No.: US 2013/0118995 A1
(43) Pub. Date: May 16, 2013

(54) SOLAR-ACTIVATED PHOTOCHEMICAL PURIFICATION OF FLUIDS

(71) Applicants: R. Thomas Hawkins, II, Aloha, OR (US); Mark D. Owen, Beaverton, OR (US)

(72) Inventors: R. Thomas Hawkins, II, Aloha, OR (US); Mark D. Owen, Beaverton, OR (US)

(73) Assignee: UVCleaning Systems, Inc., Beaverton, OR (US)

(21) Appl. No.: 13/811,615

(22) PCT Filed: Jul. 22, 2011
(86) PCT No.: PCT/US11/45089

§ 371 Date: Jan. 22, 2013

Related U.S. Application Data
(60) Provisional application No. 61/367,305, filed on Jul. 23, 2010.

(51) Int. Cl. B01J 35/00

(52) U.S. Cl. CPC B01J 35/004
USPC 210/748.14; 210/251; 422/186; 422/119

(57) ABSTRACT

Disclosed herein are embodiments of a solar-activated photochemical fluid treatment system, some of which comprise a fluid vessel, a porous enclosure positioned inside of the fluid vessel, a porous enclosure positioned inside of the fluid vessel, a fiber substrate contained within the enclosure, and a semiconductor photocatalyst coupled to the fiber substrate. The fluid vessel can be configured to contain a fluid in contact with the photocatalyst such that the fluid treatment system, responsive to solar radiation applied to the photocatalyst and to the fluid in the vessel, induces photochemical modification of contaminants and living organisms in the fluid. Related methods are also disclosed.

### CROSS REFERENCE TO RELATED APPLICATION

This application claims priority to and the benefit of U.S. Provisional Patent Application No. 61/367,305, filed on Jul. 23, 2010, which is herein incorporated by reference in its entirety.

### TECHNICAL FIELD

This disclosure relates to the purification of a fluid, such as water, and more particularly to the removal, reduction and/or detoxification of contaminants in the fluid, such as organic chemicals, inorganic chemicals, heavy metals, microorganisms and others through sunlight-activated photochemical means.

### SUMMARY

In this disclosure, it is to be understood that the terms “a”, “an” and “at least one” encompass one or more of the specified elements. That is, if two of a particular element are present, one of these elements is also present and thus “an” element is present. The phrase “and/or” means “and”, “or” and both “and” and “or”. Further, the term “coupled” generally means electrically, electromagnetically, and/or physically (e.g., mechanically or chemically) coupled or linked and does not exclude the presence of intermediate elements between the coupled or associated items absent specific contrary language. Unless specifically stated otherwise, processes and methods described herein can be performed in any order and in any combination, including with other processes and/or method acts not specifically described. The exemplary embodiments disclosed herein are only preferred examples of the invention and should not be taken as limiting the scope of the invention.

Photochemical processes comprise a range of light-activated chemical reactions that have broad application in purification of fluids. A variety of these photochemical processes can be activated by sunlight. Light-activated photocatalytic oxidation is an advanced oxidation process that involves the creation of nonselective, strongly oxidizing hydroxyl radicals at the fluid-photocatalyst interface that mineralize (i.e., convert to carbon dioxide, water, and inert byproducts) a wide range of organic compounds in water or in the presence of water. The photocatalytic process also produces reduction sites that participate in reduction of inorganic ions as well as photoadsorption of toxic heavy metals. Still further, the photocatalytic process also produces “super oxygen” ions and other species that contribute to further fluid purification reactions. Semiconductor chalcogenides (particularly oxides and sulfides) namely TiO₂, ZnO, WO₃, CeO₂, ZrO₂, SnO₂, CdS, and ZnS, have been evaluated for photocatalytic effectiveness, with anatase titania (TiO₂) generally delivering the best photocatalytic performance with maximum quantum yields. Titania is known to have strong sorption affinities for heavy metals, including toxic metals such as lead, arsenic and mercury. Photoadsorption is one example of a photo-enhanced sorption process that can efficiently remove heavy metals dissolved in a fluid to stable sorption sites on the surface of a photoactivated semiconductor material. As yet another example of a photochemical process, illumination of a fluid such as water or air with light, especially with ultraviolet (UV) light, can directly induce breaking of chemical bonds through photolysis within some first organic compounds in the fluid, forming new compounds and thereby reducing the concentration of said first organic compounds. As still another example, illumination of a fluid such as water or air with light, especially UV light, of sufficient intensity can be used to disinfect the fluid photochemically by directly killing or sterilizing microorganisms therein. As yet another example, illumination of a fluid such as water or air with light of sufficient intensity can disinfect the fluid indirectly by photothermally heating the fluid and thereby killing microorganisms therein. A plurality of photochemical processes, such as selected from the group comprising photocatalytic oxidation, photocatalytic reduction, photolysis, photodisinfection, photoadsorption and photothermal disinfection, as well as other photo-activated processes, acting synergistically, can be used in the optimization of photochemical treatment systems.

One aspect of embodiments of the present disclosure is the enabling of multiple photochemical processes in a solar-activated photochemical fluid treatment system. A further aspect of selected embodiments of the present disclosure is optimizing the performance of each photochemical process enabled in a photochemical fluid treatment system to maximize synergies among the processes. A still further aspect of selected embodiments of the present disclosure is the improvement of mass transport of contaminants in the fluid to the surface of a photocatalyst within the fluid through the enhancement of convective flow of the fluid within the treatment system. A still further aspect of selected embodiments of the present disclosure is the use of a photocatalyst coated onto or otherwise adhered to a stationary substrate within a fluid treatment vessel to effect photochemical processes for purifying fluid within the vessel. A still further aspect of selected embodiments of the present disclosure is the use of an internal mechanism within the fluid vessel to retain the photocatalyst and thereby keep it within the vessel during filling, emptying and other operations.

Photochemical processes at photocatalyst surfaces involve the illumination of the semiconductor photocatalyst with photon energies at or above the band gap energy of the semiconductor in order to create the electron-hole pairs that effect photochemical reactions at or near the semiconductor surface. Solar radiation incident on the Earth's surface comprise a broad spectrum of wavelengths, including ultraviolet (UV), visible and infrared (IR) wavelengths. A number of semiconductor photocatalyst materials, including titania (TiO₂) in its anatase structure, have band gap energies that correspond to wavelengths of light present in this solar radiation incident on the Earth's surface, and photocatalytic processes at photocatalyst surfaces can therefore be activated by this solar radiation. Solar radiation in various wavelength bands can also contribute to the activation of other photochemical processes, including but not limited to direct photodisinfection of microorganisms in the fluid and indirect disinfection through photothermal heating of the fluid.

Photochemical purification processes, including photolysis, photodisinfection, photoadsorption and photocatalysis, can require delivery of light and contaminants to reaction sites. Mass transport limits can result in practical limits on both illumination flux and photochemical reaction rates. Therefore, an exemplary approach that optimizes photochemical removal of contaminants from a fluid can involve maximizing the mass transport of contaminant species to adsorption sites on the photocatalyst material in such a photochemical system. Maximizing available photocatalyst surface area can also be desirable for an improved photochemical fluid decontamination system. In addition, flow of the fluid adjacent to a photocatalyst surface can also be desirable to improve mass transport of contaminants from the fluid to the surface. Inducing and maximizing turbulence in fluid flow near the photocatalyst surface can be a desirable aspect of a method involving a photochemical fluid decontamination system.

Suspensions of photocatalyst nanoparticles in a fluid can provide a high photocatalyst/fluid contact surface area. Nanoparticle suspensions can have, for example, surface area densities up to approximately 50 square meters per liter of treated fluid. However, suspended particles can be effectively stationary relative to the fluid, limiting fluid flow near the semiconductor-fluid interface and thereby limiting mass transport of contaminants to the surface. Additionally, a nanoparticle slurry system can require that the nanoparticles be introduced into the fluid prior to processing and then removed from the fluid after processing. A exemplary treatment system in accordance with an aspect of this disclosure improves on these nanoparticle slurry limitations by, for example: (1) permitting or inducing microscopic turbulence in flow over a photocatalyst bonded to a stationary substrate within the fluid treatment vessel, and (2) retaining the catalyst on its stationary substrate within the fluid vessel during use without requiring active management of the photocatalyst to preserve its effectiveness.

A need therefore exists for a solar-activated photochemical fluid treatment system that provides improved photochemical process rates and efficiencies and desirably without requiring active systems for photocatalyst management.

Some aspects of the present disclosure relate to an apparatus and method for fluid treatment that employs one or more photochemical mechanisms to provide efficient removal of multiple contaminants from the fluid. Exemplary embodiments can incorporate at least one treatment vessel containing a photocatalyst on a fixed porous substrate within the vessel. Such embodiments can have a fluid inlet to the treatment vessel and a fluid outlet from the treatment vessel. The inlet and the outlet can be the same opening. The inlet and/or outlet can incorporate closure mechanisms, such as valves or covers, to secure the contents of the treatment vessel during storage, transport and operation. Furthermore, the inlet and/or outlet can incorporate filtration mechanisms, such as particulate filters, in the fluid flow path.

Exemplary embodiments desirably treat fluid within the vessel by irradiating the fluid and photocatalyst with solar radiation. The vessel can comprise an at least partially sunlight transmissive portion, such as a clear plastic portion or window, to transmit solar radiation into the vessel and to the photocatalyst.

Exemplary embodiments can treat the fluid in a flowing state, wherein fluid flows from the inlet to the outlet during the treatment process, or in a stationary (batch) state, wherein the fluid is retained within the treatment vessel during the treatment process. In a stationary fluid treatment process, the fluid inlet and fluid outlet can comprise a single port for fluid flow both into the vessel prior to treatment and out of the vessel after treatment.

Exemplary embodiments disclosed can be efficient. Exemplary embodiments can enable a plurality of photochemical processes to act synergistically in a single apparatus. One or more of the following features can be included in exemplary embodiments: features that improve mass transport of contaminants to photocatalyst surfaces within the treatment vessel such as through the use of a randomly oriented, narrow fiber photocatalyst substrate, with resulting increase in photochemical process rates; features that enhance convective flow of the fluid within the treatment vessel by directly heating at least one portion of the treated fluid by absorbed solar radiation or by indirectly heating at least one portion of the treated fluid by directly heating at least one surface of the treatment vessel adjacent the treated fluid by absorbed solar radiation; and features that enhance the optimization of the amount and distribution of photocatalyst within the photochemical fluid treatment vessel to maximize process rates.

In some embodiments, photocatalyst can be placed in one or more containers, such as one or more bags (that can be small or large and that can operate in the same manner as tea bags), which can comprise buoyant material or can be supported by buoyant material, such that the container can float within a fluid to be treated so as to position the photocatalyst near a sunlight source, and near to the top of the chimney of thermal convection.

The foregoing and other features of the invention will become more apparent from the following detailed description, which proceeds with reference to the accompanying figures.

### BRIEF DESCRIPTION OF THE DRAWINGS

FIG. 1 is a side elevational view of an embodiment in accordance with the present disclosure.

FIG. 2 is a side elevational view of another embodiment of in accordance with the present disclosure.

FIG. 3 is a view of an enclosure containing fibrous material that can be included within a fluid vessel in an embodiment in accordance with the present disclosure.

FIG. 4 is a side elevational view of an embodiment in accordance with the present disclosure with FIG. 3 enclosure included therein.

FIG. 5 is a perspective view of an embodiment in accordance with the present disclosure comprising handles, handle openings, and/or handle straps.

FIG. 6 is a view of an embodiment in accordance with the present disclosure.

### DETAILED DESCRIPTION

In accordance with desirable embodiments, one or more photocatalysts can be affixed to or coupled to, such as bonded to, a fibrous substrate in a solar-activated photochemical reactor apparatus and method for the disinfection and purification of a fluid, such as water or air, for use in commercial and industrial applications. Applications include, but are not limited to, point-of-use markets, for cleanup of contaminated process outflow such as waste water and exhaust gases, and environmental remediation. Of course these are just examples and one skilled in the art will recognize a wide range of additional applications of the present disclosure, including, but not limited to, producing drinking water or process water and removing biological oxygen demand and total organic carbon from waste water and greywater. Transportable embodiments are also useful for remote applications such as purification of water in the developing world, for crisis response, or for hiking, boating, or as an emergency back-up purification system.

An effective and efficient solar-activated photochemical system for fluid disinfection and purification with photocatalytic functionality can utilize the delivery of sufficient solar illumination intensity to a photocatalyst to activate its photochemical performance, and the incorporation of sufficient photocatalyst to effectively absorb that light. Furthermore, the illuminated photocatalyst can be dispersed or distributed within at least a portion of the fluid being treated in order to purify and disinfect substantially all, or all, the fluid effectively. Still furthermore, contaminants in the fluid can be substantially, if not entirely, purified and disinfected at the surface of the photocatalyst, so that it can be desirable that the surface area of the photocatalyst is relatively large. It can also desirable that contaminants be delivered to that surface through mass transfer induced by fluid flow over the photocatalyst surface. It can be still further desirable that this mass transfer is further enhanced by inducing turbulent fluid flow over the photocatalyst surface. Additionally, the photochemical processes involved can be accelerated by temperature increase, so it can be further desirable to heat the fluid during the process if the resulting warmed fluid (if not thereafter cooled) is acceptable for its final use.

In some exemplary embodiments for disinfecting and purifying a fluid, the fluid to be treated can be presented to, or exposed to, an inert, semi-rigid, fibrous material that is at least partially transmissive to light, such as sunlight (i.e., the fibrous material allows at least a portion of sunlight incident upon it to pass into and/or through the fibrous material), and through which fluid can flow, and onto which one or more high-surface-area photocatalysts can be permanently bonded. The terms “sunlight”, “solar light”, “solar radiation”, “solar illumination” and the like are used interchangeable herein. The terms “transmissive to sunlight”, “sunlight transmissive”, and the like can be defined with respect to specific sunlight wavelengths, such as a spectrum of UV sunlight that is between 350 nm and 400 nm. A fibrous material is defined to be partially transmissive to sunlight if at least 30% of the sunlight in the 350 nm to 400 nm spectral range incident on the fibrous material penetrates to a depth of 1 cm into the fibrous material. The light transmissivity is affected not only by the material forming the fibers, but also by the packing density thereof. A material is defined to be light transmissive (e.g., a material for an overall bag or enclosure, or individual fibers of a firbrous material) if at least 30% of the sunlight in the 350 nm to 400 nm incident on the material passes through the material. In this disclosure, the term substantially transmissive to sunlight means greater than 70% transmissive of sunlight in the 365 nm to 390 nm range and greater than 80% transmissive of sunlight in the 400 nm to 1000 nm range.

Embodiments of the photocatalyst material described in the present disclosure and the exemplary apparatuses and methods for its use in photochemical disinfection and purification of fluids can be further characterized by high mass transfer efficiency resulting from fluid flow through the photocatalyst material with low pressure drop in a flow-through configuration. Embodiments of means for effecting fluid flow through an inert, semi-rigid, fibrous material onto which one or more high-surface-area photocatalysts are permanently bonded, and further for effecting fluid flow through this material, can be characterized by the use of selective absorption of solar radiation within the fluid, or otherwise within or exterior to the fluid treatment vessel, to enable and/or to enhance convective flow of the fluid within the fluid treatment vessel, especially for batch treatment processes.

Some desirable embodiments can comprise a photocatalyst bonded to a narrow, at least partially sunlight transmissive fiber substrate material to provide improved photocatalytic performance. The substrate material can be, for example, quartz, glass or another ceramic, or it can be a polymer or other plastic that can be readily formed into fiber. The photocatalyst can be selected, for example, from the semiconductor chalcogenides including TiO₂. Some embodiments employ titania (titanium dioxide, TiO₂) nanoparticle material for the photocatalyst coating because of its established effectiveness in photocatalytic degradation of organic materials, and quartz fiber for the substrate because titania bonds particularly well to quartz. Some embodiments further employ a specific surface area density of >500 m² per gram of photocatalyst.

One exemplary embodiment comprises a coating of TiO₂ on a loosely woven silica fiber substrate, prepared so that a majority (more than 50%) of the TiO₂ is in its anatase form and so that the specific surface area of the coating is approximately 1000 times the surface area of the fiber substrate, and the coating thickness is less than one micron. Quartzel® is a commercially-available example of such a substrate with TiO₂ adhered thereto and is available from Saint-Gobain.

The fiber substrate can be prepared as a mass of fibers with random fiber orientation and spacing. The mass distribution of the photocatalyst can therefore be determined by the thickness of the photocatalyst coating, the diameter of the fibers comprising the substrate, and the density of the fiber mass. For example, with a 9 μm fiber diameter and a 0.5 μm coating thickness, and with approximately 100 m of this coated fiber per mL of volume, the specific photocatalyst area density can be greater than 2000 m²/L. The fiber mass in this example comprises approximately 1% of the volume it occupies, so that the fiber mass presents low impedance to fluid flow and therefore a low fluid pressure drop in flow across the fiber mass. The fiber-to-fiber spacing in this example varies from zero to more than 1 mm, with average spacing of approximately 0.5 mm, presenting a wide range of effective pore sizes and diverging pathways to fluid flowing through the fiber mass.

In an application where a fluid flows through a treatment vessel containing such a fiber substrate coated with photocatalytic material, this tortuosity of flow paths can result in microturbulence that disrupts the flow as well as the boundary layer at the photocatalyst surface, and can thereby improve mass transport of contaminants in the fluid to the reactive photocatalyst surface. Macroscopic screens, woven meshes and reticulated or foam structures can be less desirable because, in many cases, they cannot achieve the tortuosity and porosity of this fibrous embodiment.

Moreover, a substrate fiber mass can be readily compressed, so that tortuosity and microturbulence within the fiber mass can be increased by compressing an appropriate quantity of the photocatalyst fiber material into a fluid containment vessel. Through this process, the mean fiber spacing and the resulting porosity of the fiber mass can be adjusted to optimize the flow of fluid across photocatalyst surfaces within the fluid.

Furthermore, in one example, the fibrous material can comprise or consist of a quartz or other fiber substrate that is highly transmissive to sunlight over a wide range of wavelengths useful for creating electron-hole pairs in multiple photocatalyst systems. This transmissivity provides pathways through the substrate for sunlight to penetrate to the photocatalyst coating even in the presence of strong optical absorption by contaminants in the fluid being treated.

In some embodiments, photocatalyst coated onto a fibrous substrate can be captured and contained in a porous bag or enclosure that permits fluid flow through the enclosure and allows sunlight to pass through the enclosure to activate the photocatalyst. One exemplary enclosure material is an open mesh made of a heat-sealable polymer or other plastic material. The term “porous” means that the enclosure can comprise sufficiently small pores or openings to mechanically contain the fiber substrate while having sufficiently large enough pores to allow the fluid to flow into and through the enclosure, and permit transmission of UV light to and through the photocatalyst coated fibers therein. The photocatalyst/fiber material can be inserted through a suitable opening into a partially formed enclosure of such mesh and then the opening can be sealed to capture the photocatalyst/fiber within the enclosure formed. Alternatively, the mesh material can be placed on either side of a photocatalyst/fiber mass and the mesh material on the opposing sides can then be heat sealed, welded or otherwise bonded around the perimeter of the photocatalyst/fiber mass.

In some embodiments, this seal of the enclosure material around the photocatalyst/fiber mass can overlap the edges of the mass, capturing the mass so that it cannot mechanically collapse to fill less than a desired portion of the enclosure and thereby have reduced photochemical interactions with fluid passing through the mass. Furthermore the enclosure material and construction methodology can be selected to create a photocatalyst/fiber filled bag that is flexible and that can therefore be easily rolled or folded to fit into a pre-formed solar fluid treatment vessel having an opening smaller than the size of the unfurled enclosure.

Still furthermore, the enclosure material and construction methodology can be selected to create a photocatalyst/fiber filled bag that has an overall density near or below the density of the fluid being treated, so that the photocatalyst/fiber containment enclosure tends to float in, or rise toward the top of, the treatment vessel, increasing and/or maximizing the amount of UV solar radiation entering the enclosure to activate the photocatalyst inside. The enclosure material can comprise buoyant material, such as floats, such that the photocatalyst can float and/or can be positioned in fluid being treated near the upper surface of such fluid. In some embodiments, the enclosure can remain near the center of the fluid vessel due to the geometry of the vessel and the enclosure. Alternatively, the treatment vessel can comprise one or more supports that position the enclosure therein at a desired location within the treatment vessel. As yet another alternative, the enclosure can be coupled to or affixed to the treatment vessel to hold it at a desired location therein.

In some embodiments, food coloring or other dye can be added to the fluid being treated within the fluid treatment vessel to provide a visual indication of progress and/or completion of a purification process within the vessel. For example, the dye can gradually lighten or fade away as the purification process progresses. In some embodiments, the dye can comprise Brilliant Blue FCF dye, which comprises an organic chemical. The fading of the dye from blue to clear can serve as an indicator of the treatment of other organic elements within the fluid, as well as an indicator of overall treatment of the all contaminants in the fluid. In some embodiments, the die can bleach from blue to clear in about 2-4 hours when the treatment vessel is exposed to full sunlight. The bleaching time can vary based on the strength of the incident sunlight and other variables.

The treatment time of the fluid can vary based on many variables, such as total volume of fluid, fluid to photocatalyst ratio, strength of incident sunlight, ambient temperature, positioning/orientation of the treatment vessel, amount/density of contaminants in the fluid, etc. In general, no minimum amount of incident sunlight is required to complete the treatment processes, but the processes can be completed faster with more or stronger sunlight.

In some embodiments, the treatment vessel can be formed from or comprise rigid or flexible sunlight transmissive materials, or combinations of such materials, including quartz, glass, ceramic and/or a wide range of polymers such as nylon, polyurethane, polyethylene, polyester or blends or laminates involving these compounds or other polymer materials. In one embodiment, the treatment vessel can comprise a laminate comprising a layer of biaxially oriented nylon, such as 25 μm thick, and a layer of polyethylene, such as 165 μm thick. At least one surface of the treatment vessel can be exposed to sunlight, such as the top surface of the treatment vessel, through which solar radiation can be admitted to the photocatalyst within. The sunlight transmissive materials can comprise a window or other sunlight transmissive portion of a flexible bag or other vessel. This at least one surface or portion thereof can be at least partially transmissive to solar UV light and desirably remains at least partially transmissive after extended outdoor use and exposure to sunlight. Other surfaces of the vessel can be at least partially sunlight transmissive as well, and/or they can comprise materials that absorb sunlight and thereby directly heat the fluid within the vessel, and/or they can be coated with, on or near optically absorbent materials that absorb sunlight and indirectly heat the vessel and the fluid within it.

In some embodiments, the treatment vessel can be a fluid treatment device large enough to contain more fluid than is needed for immediate use. Such a large treatment vessel can incorporate channels to route fluid through an extended path with an influent port at one end and an effluent port at the other end of this flow path. Influent contaminated fluid can traverse this extended path, and thereby receive extended solar-activated photochemical treatment, before being withdrawn through the effluent port for use. Furthermore, the treatment vessel can be tilted in such a manner that fluid flow through the vessel can be compelled and/or assisted by gravity. Still further, fluid flow can be regulated by a valve or other mechanism at any point in the extended flow path, such as at or near the treatment vessel's effluent port, to permit extraction of treated fluid on demand.

In some embodiments, a solar fluid treatment vessel can be placed on or above another device, such as a photovoltaic array or a solar fluid heater, that can use the ultraviolet, visible and/or infrared solar radiation not absorbed by the photocatalyst within the fluid treatment vessel.

In some embodiments, the photocatalyst coating on a fiber substrate, such as quartz, glass or polymer fiber, can be enhanced by electroless or otherwise plating of a metal onto the photocatalyst in order to improve the performance of the photocatalyst in disinfection, to increase the range of light absorption, to improve the catalytic activity of the catalyst, and/or to enhance other photochemical fluid treatment processes. Exemplary photocatalysts can comprise metal chalcogenide semiconductors, including metal oxides such as titania, which exhibit good adhesion to quartz and ceramics. Electroless plating of metals onto such semiconductor coatings after the semiconductor is bonded or coupled to the fiber substrate can avoid compromising the strength of the semiconductor-fiber bond while allowing accurate control of the amount of metal added. Other methods of applying particles into the catalyst nanoparticle matrix can also be compatible with this invention, as would be apparent to those skilled in the art.

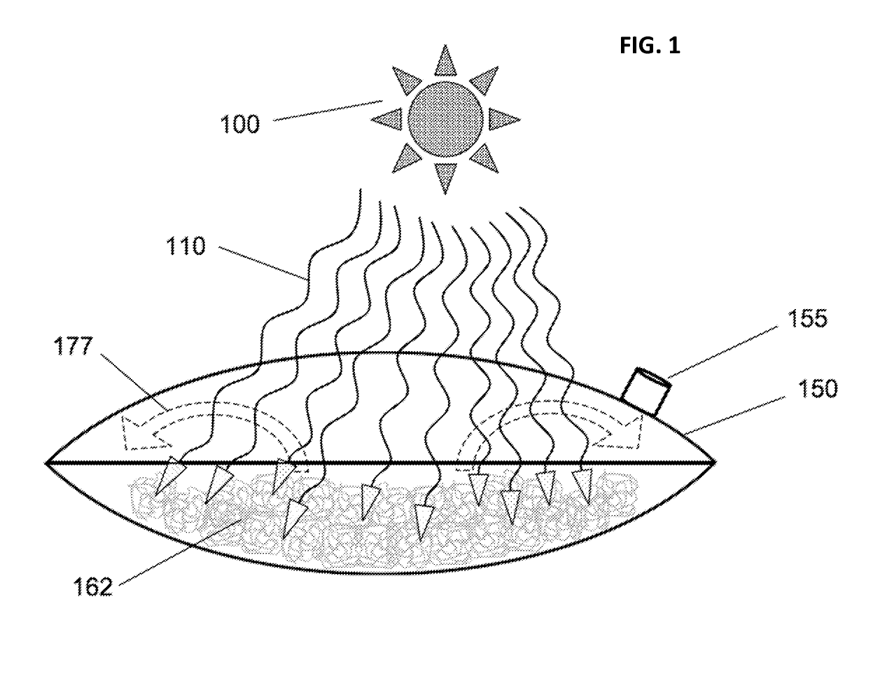

Referring now to an exemplary embodiment, FIG. 1 is a side elevational view of one form of a solar-activated photochemical treatment system schematic in accordance with the present disclosure. Electromagnetic radiation 110 from the sun 100 illuminates at least a portion of the fluid and the photocatalyst on a fiber substrate 162 within fluid treatment vessel 150. The substrate 162 can be stationary. At least a portion of this solar radiation is absorbed by at least a portion of the photocatalyst and/or directly by the contaminants in the fluid, inducing photochemical reactions that beneficially remove or otherwise detoxify contaminants present in the fluid. The semiconductor photocatalyst strongly absorbs a portion of solar radiation with wavelengths shorter than the band gap wavelength. Absorbed solar energy heats the photocatalyst, the vessel and the fluid, and nonuniformities in this heating process result in convective currents 177 within the fluid. These convective currents serve to move the fluid through the stationary substrate 162 and thereby to improve transport of contaminants in the fluid to the activated surface of the photocatalyst on the stationary substrate. The fluid treatment vessel can be fabricated from or comprise one or more flexible or rigid materials such as polymers or other plastics with at least one portion of the vessel being substantially, or at least partially, transmissive to the portion of the solar spectrum that activates the photocatalyst. At least one inlet/outlet port 155 on the fluid treatment vessel provides or comprises means for introducing fluid into the vessel for treatment and/or for removing fluid from the vessel following treatment. The at least one inlet/outlet port can incorporate or have attached at least one particle filter to remove particles from an influent fluid stream into the treatment vessel and or to remove particles from an effluent stream from the treatment vessel. More than one inlet/outlet port can be incorporated into the vessel in order to provide for flow into at least one port and out of at least one additional port so that fluid can be treated during flow through the vessel. Alternatively, a single port or opening can be used as both the inlet port and the outlet port.

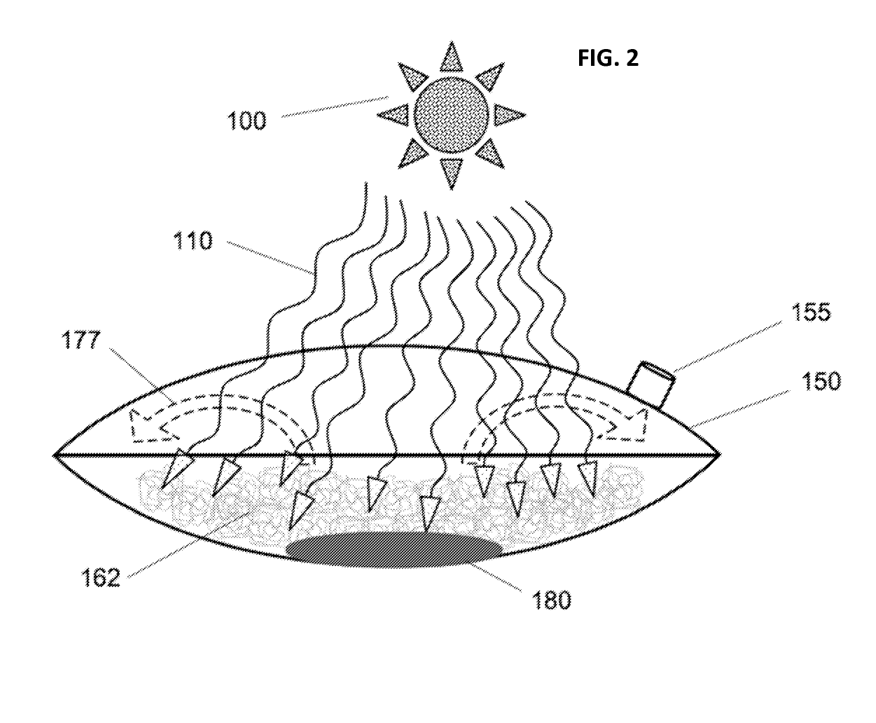

Referring now to a second exemplary embodiment, FIG. 2 is a side elevational view of a solar-activated photochemical treatment system schematic in accordance with the present disclosure. Electromagnetic radiation 110 from the sun 100 illuminates at least a portion of the fluid and the photocatalyst on a substrate 162 within the fluid treatment vessel 150. Again, the substrate 162 can be stationary. At least a portion of this solar radiation is absorbed by at least a portion of the photocatalyst and/or directly by the contaminants in the fluid, inducing photochemical reactions that beneficially remove or otherwise detoxify contaminants present in the fluid. The photocatalyst strongly absorbs a portion of solar radiation with wavelengths shorter than the band gap wavelength. Another portion of this solar radiation, including that portion that has wavelengths longer than the band gap wavelength of the photocatalyst, passes through and/or around the photocatalyst and is absorbed by an optically absorbent material 180 within, on or exterior to the vessel. This absorbed solar energy heats the optically absorbent material, and this heated material in turn heats at least a portion of the fluid within the vessel, increasing convective current flows 177 within the fluid. These convective currents serve to move the fluid through the stationary substrate 162 and thereby to improve transport of contaminants in the fluid to the activated surface of the photocatalyst on the stationary substrate. Improved transport of contaminants to the activated photocatalytic surface can increase the rate of photochemical reactions that remove or otherwise detoxify these contaminants.

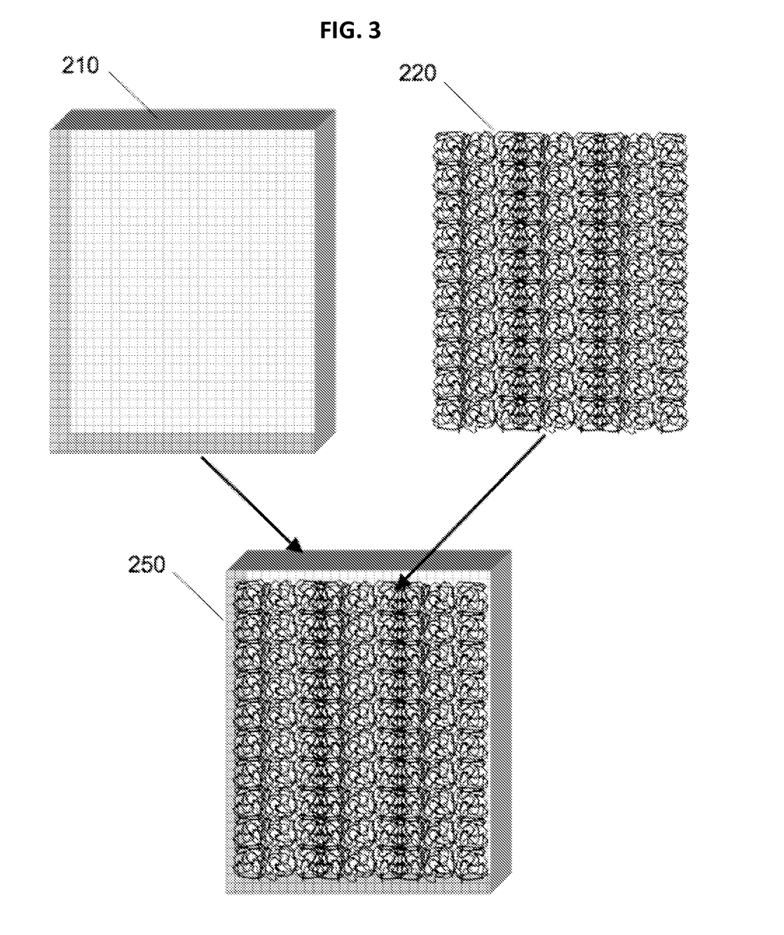

Referring now to a third exemplary embodiment, FIG. 3 illustrates schematically an enclosure, housing, or photocatalyst module, 250 comprising a mass of photocatalyst 220 (e.g., a fibrous substrate such as described above with photocatalyst carried thereon) captured within housing 210. The photocatalyst housing can be made of a material that is porous to the fluid being treated so that the fluid can readily pass through the housing during normal operation. In addition, the housing can be constructed so that sunlight can readily pass through the housing and into the photocatalyst. The module can have an overall density less than that of the fluid, in which case the photocatalyst module can float near the top of the fluid in the vessel. Materials well suited for construction of this exemplary housing can include woven or otherwise formed plastic fabric, webbing, mesh or other material that can be readily formed into suitable shapes and joined together or sealed (while still allowing contact by the photocatalyst with the fluid to be treated) to capture the photocatalyst material within the housing. Sealing the housing to capture the photocatalyst material can be accomplished by a number of means, including ultrasonic welding and heat sealing. Another mechanism for capturing the photocatalyst material within the housing involves capturing the edges of the photocatalyst material so that the shape of the photocatalyst material is preserved by the structure of the housing and does not clump into only one portion of the housing during flow of fluid into or through the housing. One approach for capturing the photocatalyst is to seal at least a portion of the housing material through edges of the photocatalyst and/or substrate material. In some embodiments, the housing material can be flexible, so that the housing containing the photocatalyst can be rolled or otherwise formed for insertion into a fluid treatment vessel, or distorted by handling or filling of the fluid vessel, without damage to the housing or photocatalyst. Still further, the housing material can be of or comprise a substantially elastic material, so that it returns substantially to its original form when stresses causing distortions of the housing are removed. One of ordinary skill in the art will recognize that a broad range of materials and sealing technologies can be utilized for fabricating this housing.

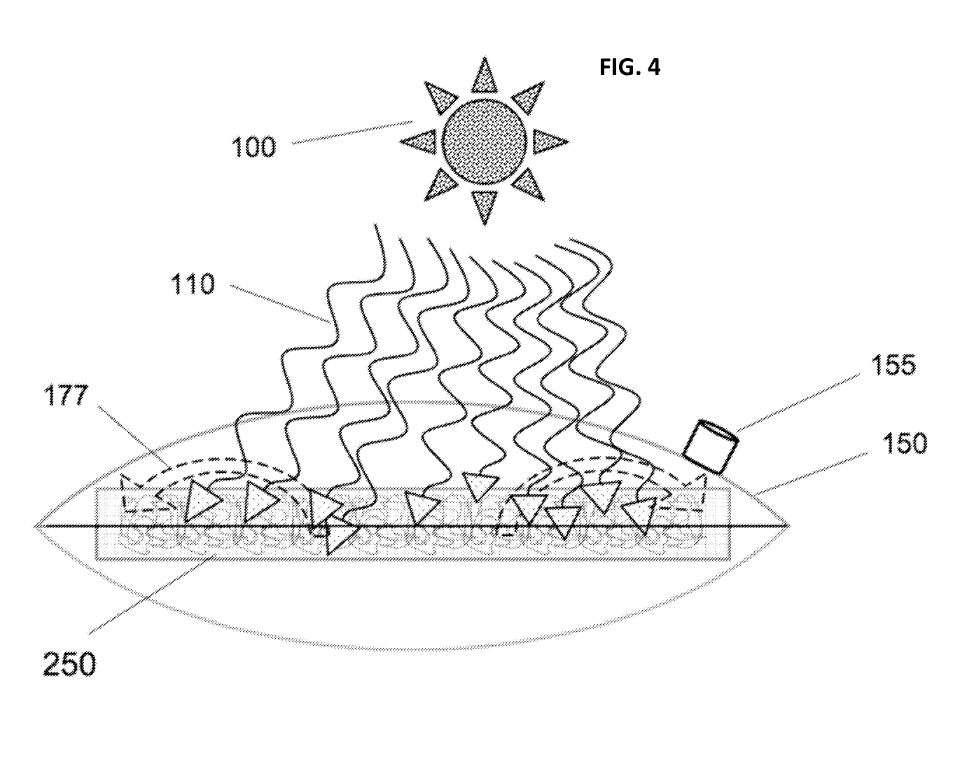

Referring now to yet another exemplary embodiment, FIG. 4 is a side elevational view of a solar-activated photochemical treatment system schematic in accordance with the present disclosure. Electromagnetic radiation 110 from the sun 100 illuminates at least a portion of the fluid and the photocatalyst on a stationary fiber substrate inside containment housing 250 within fluid treatment vessel 150. At least a portion of this solar radiation is absorbed by at least a portion of the photocatalyst and/or directly by the contaminants in the fluid, inducing photochemical reactions that beneficially remove or otherwise detoxify contaminants present in the fluid. The semiconductor photocatalyst strongly absorbs a portion of solar radiation with wavelengths shorter than the band gap wavelength. Absorbed solar energy heats the photocatalyst, the vessel and the fluid, and nonuniformities in this heating process result in convective currents 177 within the fluid. These convective currents serve to move the fluid through the photocatalyst on its substrate, which can be stationary, within an internal housing 250 and thereby to improve transport of contaminants in the fluid to the activated surface of the photocatalyst on the substrate. The fluid treatment vessel can be fabricated from or comprise one or more flexible or rigid materials such as polymers or other plastics, with at least one portion of the vessel substantially transmissive to the portion of the solar spectrum that activates the photocatalyst. The housing 250 can comprise a buoyant material such that housing 250 can float within treatment vessel 150 and/or rise toward the top of the fluid.

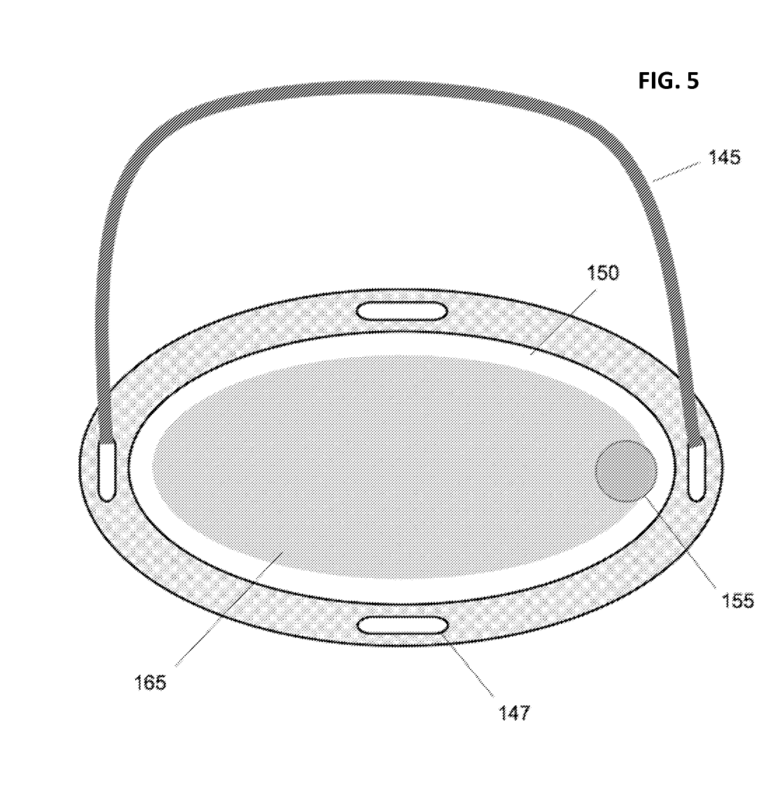

Referring now to yet another exemplary embodiment, FIG. 5 is a top view of a solar activated photochemical treatment system schematic in accordance with the present disclosure. Photocatalyst on a stationary fiber substrate 165 is contained within fluid treatment vessel 150. At least one inlet/outlet port 155 in the fluid vessel provides means for introducing fluid into the vessel for treatment and/or for removing fluid from the vessel following treatment. At least one handle 147, for example comprising a handle opening through a side seam of the treatment vessel 147 that can be reinforced, such as be a grommet ring (not shown), and/or strap 145 can be incorporated into or attached onto the fluid treatment vessel 150, such as for convenience in handling and/or to facilitate advantageous placement and/or orientation of the fluid treatment vessel for solar illumination.

In some embodiments, the fluid treatment system can be configured for ease of transportation. In some of these embodiments, the treatment system can be configured in the form of a backpack. In other embodiments, the treatment system can be configured in the form of a suitcase or briefcase, having a handle for carrying it in one hand. In some embodiments, the treatment system can comprise a grommet ring or similar holder adapted to attach the treatment system to another object, such as a backpack or tree. In some embodiments, the treatment system can comprise an at least partially sunlight transmissive upper surface and can comprise a dark or reflective lower surface. In some embodiments, both the top and bottom major surfaces of the treatment system can be at least partially sunlight transmissive.

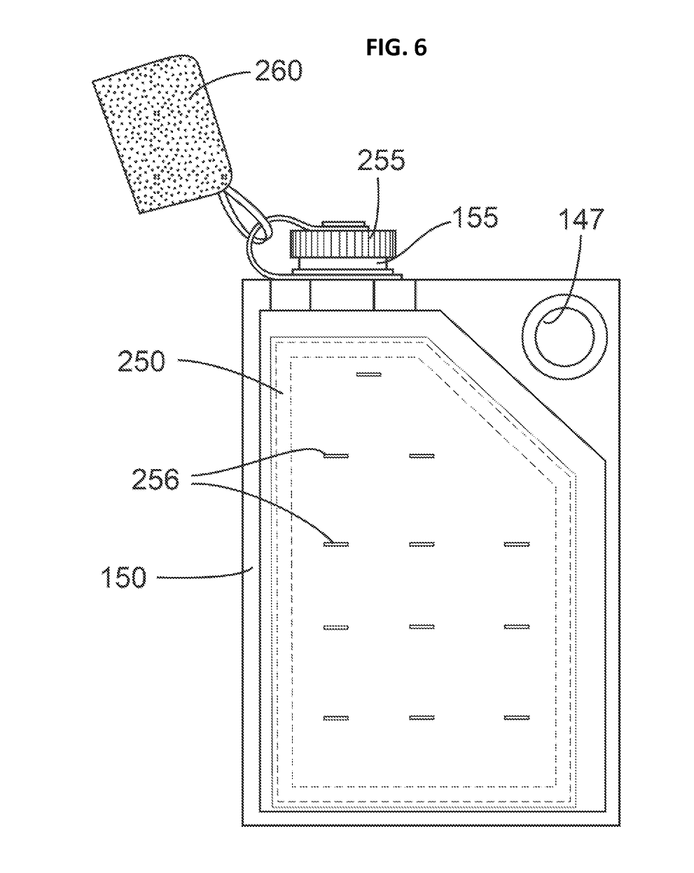

FIG. 6 shows another exemplary embodiment of a fluid treatment system. In this embodiment, the fluid vessel 150 comprises a generally rectangular, non-porous polymeric bag and the photocatalyst enclosure 250 comprises a porous mesh material that is positioned loosely within the bag 150. The bag 150 can comprise flexible nylon and/or polyethylene, for example. A clear laminate comprising 100ga biaxially oriented nylon and 6.5 mil polyethylene is one desirable exemplary material. The bag 150 can be formed by folding a sheet of the polymeric material in half and bonding the edges together, or by bonding or heat sealing two layers of the polymeric material together around the perimeter. The bag 150 can comprise opposed first and second major surfaces. At least a portion of the first major surface can be at least partially sunlight transmissive, such that when the first major surface is exposed to sunlight, at least a portion of the sunlight is admitted into the bag and to the photocatalyst. More desirably, the first major surface is substantially sunlight transmissive, or at least the portion of the first major surface covering the fluid to be treated is substantially sunlight transmissive. The second major surface of the bag 150 can also be at least partially sunlight transmissive, such that sunlight can enter the bag 150 from both sides. The first major surface can be an upper surface if the bag 150 is laid flat and facing upwardly and the second major surface can be a lower surface placed on the ground or other support. In other embodiments, the second major surface can be at least partially opaque, reflective, and/or can comprise a dark colored portion. A reflective portion of the lower surface can reflect light passing through the bag 150 and/or through the enclosure 250, such that a portion the reflected light can pass through the enclosure again and enhance the photochemical processes. A dark portion on the second major surface of the bag 150, such as in the form of writing or a logo for example, can absorb solar radiation and generate heat to create convective currents in the fluid within the bag 150, which can enhance the fluid treatment processes.

Sunlight transmissive portions of the bag 150 can allow at least some of the incident sunlight in the spectrum between 350 nm and 390 nm to be transmitted into or out of the bag, such as at least 75% of incident sunlight in this spectrum. In some examples, more than 90% of the incident sunlight in the 350 nm to 390 nm spectrum can be transmitted through sunlight transmissive portions of the bag 150. The bag 150 can comprise an opening or port 155, which can function as an inlet and an outlet for fluid, and a closure 255 for sealing the opening 155. The opening 155 can have a diameter of about 42 mm. In addition, a filter 260 can be included, such as attached to the closure 255 by a lanyard (as shown in FIG. 6), for positioning over the opening 155 for use in filtering the fluid entering or exiting the bag 150. The filter 260 can be tethered to the closure 255 or another portion of the bag 150, and the filter can be removable from and reinsertable into the opening 155.

The bag 150 can further comprise a handle 147, such as a grommet ring or other holding device, for attaching the bag to another object. For example, the handle 147 can be used to hold the bag with a hand, to attach the bag to a backpack during transportation, and/or to hang the bag from a tree branch to expose the bag to sunlight. The handle 147 can occupy one corner of the bag 150, such that the internal cavity within the bag forms a five-sided polygon, or a rectangular shape with one corner truncated, as shown in FIG. 6. Accordingly, the enclosure 250 can have the same general shape, but slightly smaller, to fit within the bag 150. The enclosure 250 can comprise a mesh fabric, such as comprised of flexible polypropylene, with the fiber substrate and photocatalyst contained therein. The mesh fabric can be folded over the substrate and heat sealed and/or sewn around the edges to enclose the substrate. Spaced apart staples or other fasteners (one being numbered 256 in FIG. 6) passing through the substrate and walls of the enclosure 250 can also be used to hold the substrate within the enclosure. In some embodiments, the enclosure 250 can have an average thickness of less than 2 cm, such as about 1 cm. When the bag 150 is filled with fluid, the thickness of the bag can expand to several inches, such as between 2 and 3 inches, or about 2.5 inches. Like a tea bag, the enclosure 150 can be free to move and/or float within the fluid within the bag 150. However, the tight-fitting geometry of the enclosure 250 within the cavity of the bag 150 (the enclosure can have about the same length-width dimensions as the bag cavity) can keep the enclosure 250 positioned at about the middle of the thickness of the bag when it is filled with fluid. In other words, when the bag 150 is filled with fluid and laid flat, the top surface of the enclosure 250 can be spaced from the upper major surface of the bag and the bottom surface of the enclosure can be spaced from the lower major surface of the bag, except that portions of the enclosure around the perimeter of the enclosure can remain in contact with the bag (see FIG. 4). Alternatively, the enclosure can be buoyant, with an overall density less than water so that it floats in the bag. As another alternative, the enclosure can be of an overall density such that it is near the density of the fluid. As a further alternative, spacers can hold the enclosure at a desired position within the bag from the major surfaces (e.g., equal distance away) or closer to a surface such as the first or upper surface. The enclosure can also be coupled to the outer bag, although in a desirable example it is loosely positioned therein. During manufacture, the enclosure can be made and rolled up or folded so as to be insertable through the opening 255 to complete the assembly of the treatment system.

In use, water or other fluid to be treated can be admitted to the bag 150 through the opening 155 and the filter 260 and into contact with the enclosure 250 and photocatalyst within the bag. The opening 155 can then be sealed with the closure 255 and the bag can be exposed to sunlight, such as by laying it out on its bottom major surface or by hanging it by the handle 147. The sunlight can pass through the bag 150 and the fluid and can interact with the photocatalyst and the fluid to treat the fluid gradually over a treatment period. Convective currents caused by uneven heating patterns in the fluid can cause the fluid to move through the enclosure, like a tea bag in a cup of hot water, where it interacts with the photocatalyst. After the treatment period, the treated fluid can be dispensed through the opening 155 and be used.

In testing, 3 liters of water in the bag can be treated in 1 to 2 hours in full midday sunlight at about 80° F., and in 2 to 4 hours on a cloudy day at about 65° F.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope of these claims.

1. A solar-activated photochemical fluid treatment system comprising:
a fluid vessel having at least one opening and comprising an at least partially sunlight transmissive portion;
at least one enclosure positioned inside of the fluid vessel, the enclosure comprising material that allows fluid and sunlight to pass into the enclosure;
an at least partially sunlight-transmissive fiber substrate contained within the at least one enclosure; and
a semiconductor photocatalyst coupled to the fiber substrate;
wherein the fluid vessel is configured to contain a fluid such that, responsive to solar radiation passing through the at least partially sunlight transmissive portion of the fluid vessel and into the at least one enclosure and to the semiconductor photocatalyst, photochemical modification of contaminants and living organisms in the fluid occurs.

2. The system of claim 1, wherein the at least one enclosure comprises a porous bag that contains the fiber substrate and the photocatalyst and allows the fluid and solar radiation to pass into the porous bag.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. The system of claim 1, wherein the at least one enclosure is not attached to the fluid vessel and can move within the fluid relative to the fluid vessel.

16. The system of claim 1, wherein the at least one enclosure has an overall density of less than or equal to a density of the fluid, such that the at least one enclosure floats or rises toward the top of the fluid in the fluid vessel.

17. The system of claim 1, wherein the at least one enclosure comprises a buoyant material such that the at least one enclosure floats or rises toward the top of the fluid in the fluid vessel.

18. The system of claim 1, wherein the at least one enclosure is foldable or rollable such that is insertable through the at least one opening in the fluid vessel and unfurlable within the fluid vessel.

19. The system of claim 1, wherein the at least one enclosure comprises a polymeric mesh.

20. The system of claim 1, wherein the mesh is comprised of a material that is at least partially sunlight transmissive.

21. The system of claim 1, wherein the fluid vessel comprises a flexible polymeric material.

22. The system of claim 1, further comprising at least one filter positionable in the at least one opening for filtering the fluid to prevent at least some particulate or other contaminants from entering or exiting the fluid vessel.

23. The system of claim 1, wherein the fluid in the fluid vessel comprises a dye that is an indicator of the purification of the fluid.

24. The system of claim 1, wherein the fluid vessel comprises at least one handle or strap for supporting the vessel during operation or transportation.

25. The system of claim 1, wherein the combined volume of the photocatalyst and the fiber substrate is less than 5% of the volume of the fluid within the fluid vessel.

26. The system of claim 1, wherein the specific surface area of the photocatalyst within at least one portion of the fluid is greater than 100 square meters per liter of the fluid.

27. The system of claim 1, further comprising a solar radiation absorber positioned external to the enclosure, the solar radiation absorber being operable to convert absorbed solar radiation into heat and to thereby heat a portion of the fluid adjacent to the solar radiation absorber and thereby drive convective circulation of the fluid within the fluid vessel.

28. (canceled)

29. A solar-activated photochemical fluid treatment system comprising:
a fluid vessel;
an at least partially sunlight-transmissive fiber substrate contained within the fluid vessel;
a semiconductor photocatalyst coupled to the fiber substrate; and
means for driving convective circulation of the fluid within the fluid vessel;
wherein the fluid vessel is configured to contain a fluid in contact with the semiconductor photocatalyst such that the fluid treatment system, responsive to solar radiation applied to the semiconductor photocatalyst, is configured to induce photochemical modification of contaminants in the fluid.

30. (canceled)

31. The system of claim 29, wherein the means for driving convective circulation of the fluid within the fluid vessel comprises optically absorbent material attached to the exterior of the fluid vessel.

32. A method for purifying a fluid by introducing the fluid into a photochemical treatment system, the method comprising:
providing at least one non-porous fluid vessel with at least one porous enclosure position within the at least one vessel;
providing a semiconductor photocatalyst coupled to an at least partially sunlight-transmissive fiber substrate that is confined within the at least one enclosure;
introducing a fluid into the at least one fluid vessel such that the introduced fluid is in contact with at least a portion of the semiconductor photocatalyst; and
admitting solar radiation into the at least one fluid vessel to illuminate at least a portion of the fluid and at least a portion of the photocatalyst within the at least one enclosure to induce photochemical modification of contaminants in the fluid.

33. The method of claim 32, further comprising modifying the photocatalyst after it is coupled to the fiber substrate.

34. The method of claim 32, wherein a metal is deposited onto the photocatalyst by an electroless process after it is coupled to the fiber substrate.

35. An enclosure apparatus for positioning inside of a fluid containing vessel that has an at least partially sunlight transmissive portion and for use in solar-activated photochemical treatment of fluid to be treated when the enclosure apparatus and fluid to be treated is placed inside of the vessel, the enclosure apparatus comprising:
an enclosure housing comprising a material that allows fluid and sunlight to pass into the enclosure;
an at least partially sunlight transmissive fiber substrate contained within the enclosure housing;
a semiconductor photocatalyst coupled to the fiber substrate;
wherein positioning of the enclosure module inside the fluid vessel with fluid to be treated, responsive to solar radiation passing through the at least partially sunlight transmissive portion of the fluid vessel and to the semiconductor photocatalyst in the enclosure housing, results in photochemical modification of contaminants and living organisms in the fluid occurs.

36. The enclosure apparatus of claim 35 which has an overall density of less than or equal to a density of the fluid to be treated, such that the enclosure apparatus can float or rise toward the top of the fluid in the fluid to be treated in the vessel.

37. The enclosure apparatus of claim 35 which has an overall density of less than or equal to a density of the fluid to be treated, such that the enclosure apparatus can float or rise toward the top of the fluid in the fluid to be treated in the vessel.

38. The enclosure apparatus of claim 35 which is foldable or rollable.

39. The enclosure apparatus of claim 35 wherein the enclosure housing comprises a polymeric mesh.

40. The enclosure apparatus of claim 39 wherein the mesh is at least partially sunlight transmissive.
